(19) United States
(12) Patent Application Publication
(10) Pub. No.: US 2025/0295457 A1
(43) Pub. Date: Sep. 25, 2025

(54) CONFIGURATION OF ROBOTIC SYSTEMS FOR ERGONOMIC STATES OF OPERATORS IN MEDICAL PROCEDURES

(71) Applicant: Intuitive Surgical Operations, Inc., Sunnyvale, CA (US)

(72) Inventors: Reza Khodayi Mehr, Sunnyvale, CA (US); Omid Mohareri, San Francisco, CA (US)

(73) Assignee: Intuitive Surgical Operations, Inc., Sunnyvale, CA (US)

(21) Appl. No.: 19/068,985

(22) Filed: Mar. 3, 2025

Related U.S. Application Data
(60) Provisional application No. 63/569,024, filed on Mar. 22, 2024.

(51) Int. Cl. A61B 34/30; A61B 34/00; A61B 34/20

(52) U.S. Cl. CPC A61B 34/30; A61B 2034/2065; A61B 2034/252

(57) ABSTRACT

Aspects of this technical solution can receive a set of data including one or more images of one or more body parts of an operator, the one or more images depicting the one or more body parts engaged with one or more components of a robotic system or instrument, the set of data corresponding to a medical procedure, and generate, based at least in part on one or more of the images, an output corresponding to a configuration of the robotic system or instrument, the output comprising one or more instructions to set or modify one or more physical positions of the one or more components.

### CROSS-REFERENCE TO RELATED APPLICATION

This application claims the benefit of, and priority to, U.S. Patent Application No. 63/569,024, filed Mar. 22, 2024, the full disclosure of which is incorporated herein in its entirety.

### TECHNICAL FIELD

The present implementations relate generally to medical devices, including but not limited to configuration of robotic systems for ergonomic states of operators in medical procedures.

### INTRODUCTION

Surgeons are increasingly expected to perform more complex medical procedures over longer durations. As the complexity and duration of medical procedures increase, physical strain on surgeons increases. This strain can cause a decrease in efficiency and accuracy of surgeons during medical procedures and increase the risk of a decrease in the efficiency and accuracy of surgeons over the long term. Due at least to variations in physiology, techniques, experience, and range of motion among surgeons, conventional systems cannot effectively and accurately accommodate individual surgeons to reduce their strain during medical procedures.

### SUMMARY

Systems, methods, apparatuses, and non-transitory computer-readable media are provided for modifying a configuration of a robotic system in response to an ergonomic state of an operator of a robotic device. Thus, this technical solution can include an ergonomically intelligent surgeon console that can adapt to the physical characteristics of a surgeon, including adapting to specific body pose, hand pose and range of motion of an individual surgeon, and automatically adjust various ergonomic settings of the console for optimal use. For example, a system can detect positions of one or more fingers, hands, wrists, or forearms of a surgeon holding or near a robotic controller. The system can determine an ergonomic configuration of a grip or posture of the surgeon based on the detected body parts and, optionally, supplemented with state information of the robotic device. For example, state information can include location, orientation, or displacement of the robotic controller, a seat, an armrest, or any other portion of the robotic device or system that can support the surgeon. The state information can provide additional data that increases the granularity of an estimate of an ergonomic position of the surgeon. Thus, a technical solution for configuration of robotic systems for ergonomic states of operators in medical procedures is provided.

At least one aspect is directed to a system. The system can include one or more processors, coupled with memory. The system can receive a set of data that can include one or more images of one or more body parts of an operator, the one or more images depicting the one or more body parts engaged with one or more components of a robotic system or instrument, the set of data corresponding to a medical procedure. The system can generate, based at least in part on one or more of the images, an output corresponding to a configuration of the robotic system or instrument, the output can include one or more instructions to set or modify one or more physical positions of the one or more components.

At least one aspect is directed to a method. The method can include receiving a set of data, which can include one or more images of one or more body parts of an operator, the one or more images depicting the one or more body parts engaged with one or more components of a robotic system or instrument, the set of data corresponding to a medical procedure. The method can include generating, based at least in part on one or more of the images, an output corresponding to a configuration of the robotic system or instrument. The output can include one or more instructions to set or modify one or more physical positions of the one or more components.

At least one aspect is directed to a non-transitory computer-readable medium, which can include one or more instructions stored thereon and executable by a processor. The processor can receive a set of data that can include one or more images of one or more body parts of an operator, the one or more images depicting the one or more body parts engaged with one or more components of a robotic system or instrument, the set of data corresponding to a medical procedure. The processor can generate, based at least in part on one or more of the images, an output corresponding to a configuration of the robotic system or instrument. The output can include one or more instructions to set or modify one or more physical positions of the one or more components.

At least one aspect is directed to a system. The system can include one or more processors, coupled with memory. The system can receive data that can include one or more images of one or more body parts of an operator, the one or more images depicting the one or more body parts engaged with one or more components of a robotic system or instrument, the set of data corresponding to a medical procedure. The system can generate, using a first model configured to detect image features, a first feature that identifies one or more physical positions of the one or more body parts. The system can generate, using a second model receiving the first feature as input, an output corresponding to the one or more physical positions of the one or more body parts. The output can include one or more instructions to set or modify one or more physical positions of the one or more components. The system can determine, based on the first feature, a loss with respect to the output. The system can update at least one of the first model and the second model based on the loss.

At least one aspect is directed to a method. The method can include receiving data, which can include one or more images of one or more body parts of an operator, the one or more images depicting the one or more body parts engaged with one or more components of a robotic system or instrument, the set of data corresponding to a medical procedure. The method can include generating, using a first model configured to detect image features, a first feature that identifies one or more physical positions of the one or more body parts. The method can include generating, using a second model receiving the first feature as input, an output corresponding to the one or more physical positions of the one or more body parts. The output can include one or more instructions to set or modify one or more physical positions of the one or more components. The method can include determining, based on the first feature, a loss with respect to the output. The method can include updating at least one of the first model and the second model based on the loss.

At least one aspect is directed to a non-transitory computer-readable medium can include one or more instructions stored thereon and executable by a processor. The processor can receive data that can include one or more images of one or more body parts of an operator, the one or more images depicting the one or more body parts engaged with one or more components of a robotic system or instrument, the set of data corresponding to a medical procedure. The processor can generate, using a first model configured to detect image features, a first feature that identifies one or more physical positions of the one or more body parts. The processor can generate, using a second model receiving the first feature as input, an output corresponding to the one or more physical positions of the one or more body parts. The output can include one or more instructions to set or modify one or more physical positions of the one or more components. The processor can determine, based on the first feature, a loss with respect to the output. The processor can update at least one of the first model and the second model based on the loss.

At least one aspect is directed to a medical system. The medical system can include a manipulator assembly configured to support one or more medical instruments; an input system configured to be operated by an operator to control the manipulator assembly, wherein the input system includes an operator workspace. The medical system can include a sensing system comprising one or more sensors, the one or more sensors having a combined field of view of at least a portion of the operator workspace. The medical system can include one or more processors, coupled with memory. The processors can receive sensor data from the one or more sensors of the sensing system. The processors can generate, based on the sensor data, one or more kinematic representations of at least a portion of a body of the operator. The processors can compute, based on the one or more kinematic representations, a plurality of values, the plurality of values representing joint angles of one or more joints of the body of the operator. The processors can determine, based on the plurality of values, an ergonomic metric for an action of the input system via the operator.

### BRIEF DESCRIPTION OF THE FIGURES

These and other aspects and features of the present implementations are depicted by way of example in the figures discussed herein. Present implementations can be directed to, but are not limited to, examples depicted in the figures discussed herein. Thus, this disclosure is not limited to any figure or portion thereof depicted or referenced herein, or any aspect described herein with respect to any figures depicted or referenced herein.

FIG. 1A depicts an example architecture of a system according to this disclosure.

FIG. 1B depicts an example environment of a system according to this disclosure.

FIG. 2 depicts an example robotic system according to this disclosure.

FIG. 3 depicts an example robotic system according to this disclosure.

FIG. 4 depicts an example center camera field of view according to this disclosure.

FIG. 5 depicts an example kinematic capture model for a digit according to this disclosure.

FIG. 6 depicts an example method of configuration of ergonomic states of operators of robotic systems for medical procedures according to this disclosure.

FIG. 7 depicts an example method of configuration of ergonomic states of operators of robotic systems for medical procedures according to this disclosure.

FIG. 8 depicts an example method of configuration of ergonomic states of operators of robotic systems for medical procedures according to this disclosure.

### DETAILED DESCRIPTION

Aspects of this technical solution are described herein with reference to the figures, which are illustrative examples of this technical solution. The figures and examples below are not meant to limit the scope of this technical solution to the present implementations or to a single implementation, and other implementations in accordance with present implementations are possible, for example, by way of interchange of some or all of the described or illustrated elements. Where certain elements of the present implementations can be partially or fully implemented using known components, only those portions of such known components that are necessary for an understanding of the present implementations are described, and detailed descriptions of other portions of such known components are omitted to not obscure the present implementations. Terms in the specification and claims are to be ascribed no uncommon or special meaning unless explicitly set forth herein. Further, this technical solution and the present implementations encompass present and future known equivalents to the known components referred to herein by way of description, illustration, or example.

A system can include the robotic device, and one or more sensors to detect one or more positions of one or more body parts of an operator of the robotic device. The robotic device or a system including the robotic device can modify location or orientation of one or more parts of the robotic device or a system including the robotic device, in response to detecting body position of the operator of the robotic device. The technical solution can thus provide a technical improvement, including at least combining effective sensing with intelligent models that capture dynamics of a robot and human physiology and identify optimal ergonomics from the captured dynamics. Because of the high granularity and narrow margin of error for a medical procedure, this technical solution is particularly advantageous for medical robots.

In some embodiments, the system can identify an ergonomic configuration of a robotic device for an individual operator of the robotic device. The system can provide a recommendation of an initial ergonomic configuration, personalize the configuration in response to user by the operation of the robotic device over time, monitor pose or grip of the operator during a medical procedure, and can modify the ergonomic configuration during the medical procedure to improve the ergonomics of the surgeon using the robotic device during the procedure. For example, a machine learning model can recommend and apply initial settings that correspond to an ergonomic configuration of a robotic device for a new operator of the robotic device. The machine learning model can be trained on vision data collected from surgeons conducting real procedures and analyzing the ergonomic risk factors involved in those operations against recommended ergonomic poses, for example, by a rapid upper limb assessment. This model provides a technical solution of an objective performance indicator (OPI) for surgeon console ergonomics. As the surgeon continues to use the system, the model can make personalized recommendations considering the current physique, posture, or technique of the surgeon. The model can also take into account the changes in technique and skill level of the given surgeon. These optimized ergonomic settings can be applied at the beginning of the next case.

In addition, the system can continuously monitor the pose or grip of the surgeon during surgery and can make incremental adjustments, recommendations, or alert the user to correct their posture and pose, based on surgeon preference. For example, the system can distinguish between flexible incremental adjustments that can be applied during surgery, and restricted incremental adjustments that can only be applied during a pause in surgery. For example, flexible incremental adjustments can include adjustments to positions of a seat, armrest, footrest, and seat back. For example, restricted incremental adjustments can include adjustments to positions of a robotic controller, or a surgeon headset. The system can make incremental adjustments or provide notifications of available incremental adjustments according to notification preferences for a medical procedure or a given surgeon (e.g., to reduce distraction of a surgeon during a given medical procedure). Thus, the system can provide a technical improvement to modify a physical configuration of a robotic device to increase ergonomic support of a given surgeon without disrupting the medical procedure for the surgeon.

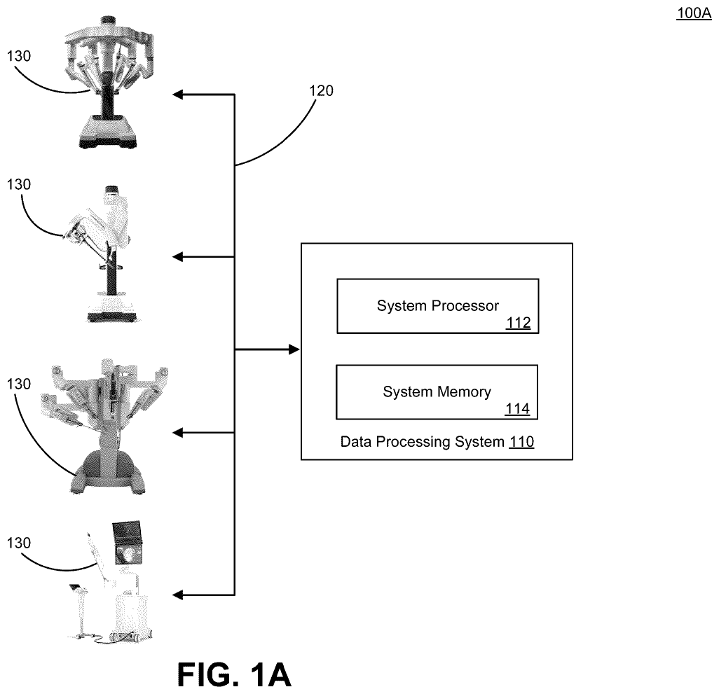

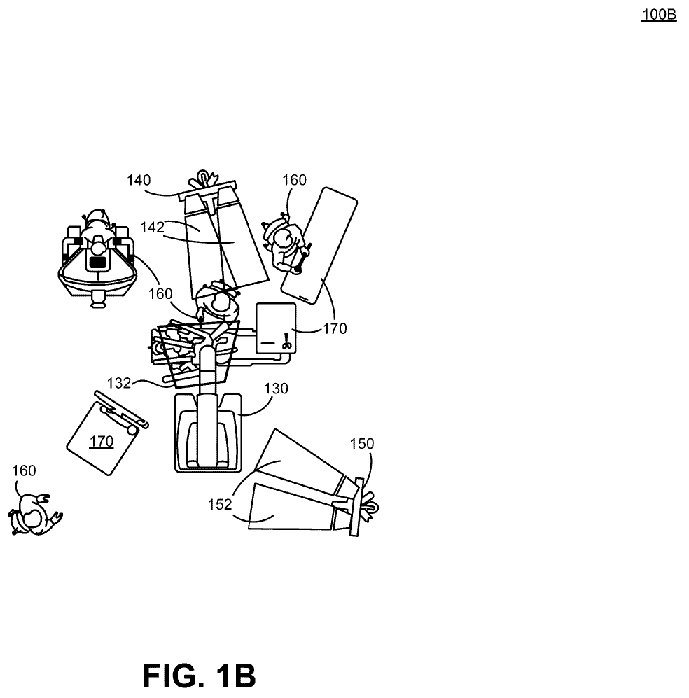

FIG. 1A depicts an example architecture of a system according to this disclosure. As illustrated by way of example in FIG. 1A, an architecture of a system 100A can include at least a data processing system 110, a communication bus 120, and a robotic system 130. In some embodiments, the system 100A can configure multiple sensors in the OR based on detection of a state or scene corresponding to the OR as a whole. The system can detect, for example, a robot docking scene, as discussed above, and can configure multiple sensors in the OR according to the field of view of the sensor or the location of the sensor in the OR. For example, the system can enter a training mode in which a model is trained with machine learning from input, including video from a plurality of camera sensors distributed within the OR. The machine learning model can optimize the configuration of the robotic system 130 for a given surgeon during a given medical procedure using a loss function that is based on at least one of the video data, parameters assigned to the surgeon, positions assigned to body parts of the surgeon at the surgeon console, or any combination thereof.

The machine learning model can treat video input from each of these sensors as a combined input for determining optimized allocation or a loss. This way, the machine learning model can be updated (e.g., trained) to provide a technical improvement to increase accuracy of configuration of a robotic system for the ergonomic state of an individual operator (e.g., a surgeon at a surgeon console of the robotic system). For example, the machine learning model can be trained with input data including one or more poses, images, videos, or any combination thereof, corresponding to one or more medical procedures. For example, a system as discussed herein can generate a trained model by the training (e.g., the data processing system 110), at a time other than during a medical procedure. For example, the data processing system 110 can obtain the trained model from an external system configured to perform the training, and can execute the trained model during a medical procedure to perform one or more actions as discussed herein. Thus, this technical solution can, by using the trained model during a medical procedure, provide a technical improvement to realize physical configurations of a robotic system responsive to state of a surgeon that varies over time within a medical procedure and across medical procedures. For example, a robotic system can modify a rotational position of a manipulator from a first angular position to a second angular position to provide a technical improvement to improve ergonomics of the surgeon (e.g., an inward grip twist of a wrist of the surgeon) without disrupting a surgery. The robotic system 130 can execute the modification of the rotation position from the first angular position to the second angular position at a rate below a predetermined threshold. Thus, the robotic system 130 can accommodate while reducing or eliminating potential disruption to the surgical activity by the surgeon in controlling the manipulators during the medical procedure.

The data processing system 110 can include a physical computer system operatively coupled or that can be coupled with one or more components of the system 100A, either directly or directly through an intermediate computing device or system. The data processing system 110 can include a virtual computing system, an operating system, and a communication bus to effect communication and processing. The data processing system 110 can include a system processor 112 and a system memory 114.

The system processor 112 can execute one or more instructions associated with the system 110. The system processor 112 can include an electronic processor, an integrated circuit, or the like including one or more of digital logic, analog logic, digital sensors, analog sensors, communication buses, volatile memory, nonvolatile memory, and the like. The system processor NNN can include, but is not limited to, at least one microcontroller unit (MCU), microprocessor unit (MPU), central processing unit (CPU), graphics processing unit (GPU), physics processing unit (PPU), embedded controller (EC), or the like. The system processor 112 can include a memory operable to store or storing one or more instructions for operating components of the system processor 112 and operating components operably coupled to the system processor 112. The one or more instructions can include at least one of firmware, software, hardware, operating systems, embedded operating systems, and the like. The system processor 112 can include at least one communication bus controller to effect communication between the system processor 112 and the other elements of the system 100A.

The system memory 114 can store data associated with the data processing system 110. The system memory 114 can include one or more hardware memory devices to store binary data, digital data, or the like. The system memory 114 can include one or more electrical components, electronic components, programmable electronic components, reprogrammable electronic components, integrated circuits, semiconductor devices, flip-flops, arithmetic units, or the like. The system memory 114 can include at least one of a non-volatile memory device, a solid-state memory device, a flash memory device, or a NAND memory device. The system memory 114 can include one or more addressable memory regions disposed on one or more physical memory arrays. A physical memory array can include a NAND gate array disposed on, for example, at least one of a particular semiconductor device, integrated circuit device, and printed circuit board device. For example, the system memory 114 can correspond to a non-transitory computer-readable medium. For example, the non-transitory computer-readable medium can include one or more instructions executable by the processor to generate, based at least in part on the one or more images, one or more models, each indicative of respective portions of the one or more body parts of the operator. The processor can generate, based at least in part on the model, the output. For example, with respect to the non-transitory computer-readable medium, the one or more physical positions of the one or more body parts each correspond to respective poses of the one or more body parts engaged with the one or more components of the robotic system or instrument.

The communication bus 120 can communicatively couple the data processing system 110 with the robotic system 130. The communication bus 120 can communicate one or more instructions, signals, conditions, states, or the like between one or more of the data processing system 110 and components, devices, or blocks operatively coupled or couplable therewith. The communication bus 120 can include one or more digital, analog, or like communication channels, lines, traces, or the like. As an example, the communication bus 120 can include at least one serial or parallel communication line among multiple communication lines of a communication interface. The communication bus 120 can include one or more wireless communication devices, systems, protocols, interfaces, or the like. The communication bus 120 can include one or more logical or electronic devices, including but not limited to integrated circuits, logic gates, flip-flops, gate arrays, programmable gate arrays, and the like. The communication bus 120 can include one or more telecommunication devices, including but not limited to antennas, transceivers, packetizers, and wired interface ports.

The robotic system 130 can include one or more robotic devices configured to perform one or more actions of a medical procedure (e.g., a surgical procedure). For example, a robotic device can include, but is not limited to, a surgical device that can be manipulated by a robotic device. For example, a surgical device can include, but is not limited to, a scalpel or a cauterizing tool. The robotic system 130 can include various motors, actuators, or electronic devices whose position or configuration can be modified according to input at one or more robotic interfaces. For example, a robotic interface can include a manipulator with one or more levers, buttons, or grasping controls that can be manipulated by pressure or gestures from one or more hands, arms, fingers, or feet. The robotic system 130 can include a surgeon console in which the surgeon can be positioned (e.g., standing or seated) to operate the robotic system 130. However, the robotic system 130 is not limited to a surgeon console co-located or on-site with the robotic system 130.

FIG. 1B depicts an example environment of a system according to this disclosure. As illustrated by way of example in FIG. 1B, an environment 100B of a system 100A can include at least the robotic system 130 having a field of view 132, a first sensor system 140, a second sensor system 150, persons 160, and objects 170. For example, the environment 100B is illustrated by way of example as a plan view of an OR having the robotic system 130, the first sensor system 140, the second sensor system 150, the persons 160, and the objects 170 disposed therein or thereabout. The presence, placement, orientation, and configuration, for example, of one or more of the robotic system 130, the first sensor system 140, the second sensor system 150, the persons 160, and the objects 170 can correspond to a given medical procedure or given type of medical procedure that is being performed, is to be performed, or can be performed in the OR corresponding to the environment 100B. This disclosure is not limited to the presence, placement, orientation, or configuration of the robotic system 130, the first sensor system 140, the second sensor system 150, the persons 160, the objects 170, or any other element illustrated herein by way of example. The field of view 132 of the robotic system 130 can correspond to a physical volume within the environment 100B that is within the range of detection of one or more sensors of the robotic system 130. For example, the field of view 132 is positioned above a surgical site of a patient. For example, the field of view 132 is oriented toward a surgical site of a patient.

The first sensor system 140 can include one or more sensors oriented to a first portion of the environment 100B. For example, the first sensor system 140 can include one or more cameras configured to capture images or video in visual or near-visual spectra and/or one or more depth-acquiring sensors for capturing depth data (e.g., three-dimensional point cloud data). For example, the first sensor system 140 can include a plurality of cameras configured to collectively capture images or video in a stereoscopic view. For example, the first sensor system 140 can include a plurality of cameras configured to collectively capture images or video in a panoramic view. The first sensor system 140 can include a field of view 142. The field of view 142 can correspond to a physical volume within the environment 100B that is within the range of detection of one or more sensors of the first sensor system 140. For example, the field of view 142 is oriented toward a surgical site of a patient. For example, the field of view 152 is located behind a surgeon at the surgical site of a patient.

The second sensor system 150 can include one or more sensors oriented to a second portion of the environment 100B. For example, the second sensor system 150 can include one or more cameras configured to capture images or video in visual or near-visual spectra and/or one or more depth-acquiring sensors for capturing depth data (e.g., three-dimensional point cloud data). For example, the second sensor system 150 can include a plurality of cameras configured to collectively capture images or video in a stereoscopic view. For example, the second sensor system 150 can include a plurality of cameras configured to collectively capture images or video in a panoramic view. The second sensor system 150 can include a field of view 152. The field of view 152 can correspond to a physical volume within the environment 100B that is within the range of detection of one or more sensors of the second sensor system 150. For example, the field of view 152 is oriented toward the robotic system 130. For example, the field of view 152 is located adjacent to the robotic system 130.

The persons 160 can include one or more individuals present in the environment 100B. For example, the persons can include, but are not limited to, assisting surgeons, supervising surgeons, specialists, nurses, or any combination thereof. The objects 170 can include, but are not limited to, one or more pieces of furniture, instruments, or any combination thereof. For example, the objects 170 can include tables and surgical instruments.

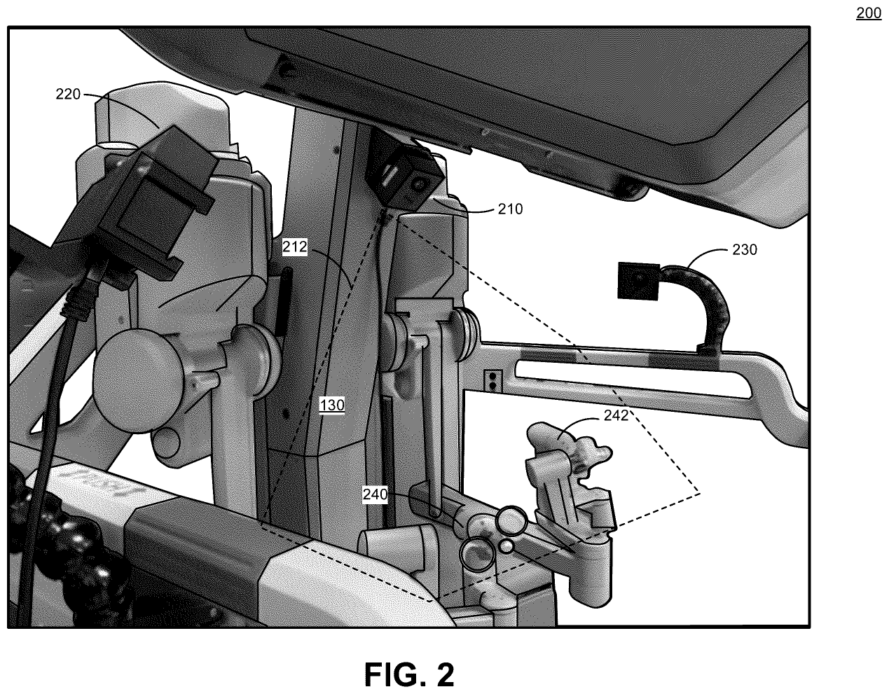

FIG. 2 depicts an example of a robotic system according to this disclosure. As illustrated by way of example in FIG. 2, a robotic system 200 can include at least a center sensor 210, a left sensor 220, a right sensor 230, a left hand manipulator 240, and a right hand manipulator 242. However, the robotic system 200 or an environment configured to capture the robotic system 200 are not limited to the sensors 210, 220 and 230 as discussed herein. For example, one or more sensors (e.g., cameras) distinct form the sensors 210, 220 and 230 can be positioned to capture body pose of an operator (e.g., surgeon) of the robotic system 200. For example, a camera can be placed to face a surgeon console and to capture images or video of a back of a surgeon, from a rear of the surgeon, one or more sides of the surgeon, or any combination thereof.

The center sensor 210 can correspond to a first sensor of the robotic system 130 at a first position of the robotic system 130. For example, the center sensor 210 can be a visible light image sensor. The center sensor 210 can also be a time-of-flight sensor or depth sensor configured to capture at least one of intensity data or depth data in one or more visual spectra or non-visual spectra (e.g., infrared or ultraviolet) that depict one or more positions of one or more body parts of a surgeon at a console of the robotic device. Alternatively, the center sensor 210 may be a multi-modal sensor that includes one or more visible light image sensor and one or more depth sensors. For example, the center sensor 210 can capture image data or video data in one or more visual spectra or non-visual spectra (e.g., infrared or ultraviolet) that depict one or more positions of one or more body parts of a surgeon at a console of the robotic device. For example, the center sensor 210 can be coupled with the robotic system 130 at a pillar or post of the robotic system facing a console of the robotic system 130. For example, a console of the robotic system 130 can include one or more manipulator devices graspable by a surgeon via the respective hands of the surgeons, and a seating area to accommodate a surgeon. The center sensor 210 can detect the image data or the video data according to a field of view 212. The field of view 212 can correspond to a physical volume within the medical environment 100B that is oriented toward the surgeon console and can be oriented to allow the center sensor 210 to detect one or more of the left hand manipulator 240 and the right hand manipulator 242. For example, the field of view 212 can be oriented to allow the center sensor 210 to detect one or more hands, fingers, wrists, arms, forearms, or any portion thereof, engaged with the robotic system 130 or at least partially within the field of view 212.

The left sensor 220 can correspond to a second sensor of the robotic system 130 at a second position of the robotic system 130. For example, the left sensor 220 can be a visible light image sensor. The left sensor 220 can also be a time-of-flight sensor or depth sensor configured capture at least one of intensity data or depth data in one or more visual spectra or non-visual spectra (e.g., infrared or ultraviolet) that depict one or more positions of one or more body parts of a surgeon at a console of the robotic device. Alternatively, the left sensor 220 may be a multi-modal sensor that includes one or more visible light image sensor and one or more depth sensors. For example, the left sensor 220 can capture image data or video data in one or more visual spectra or non-visual spectra (e.g., infrared or ultraviolet) that depict one or more positions of one or more body parts of a surgeon at the console of the robotic device. For example, the left sensor 220 can be coupled with the robotic system 130 at a left rail or arm of the robotic system facing the console of the robotic system 130. The left sensor 220 can detect the image data or the video data according to a second field of view. The second field of view can correspond to a physical volume within the medical environment 100B that is oriented toward the surgeon console and can be oriented to allow the center sensor 210 to detect the left hand manipulator 240 and optionally detect at least a portion of the right hand manipulator 242. For example, the second field of view can be oriented to allow the left sensor 220 to detect one or more hands, fingers, wrists, arms, forearms, or any portion thereof, engaged with the robotic system 130 or at least partially within the second field of view.

The right sensor 230 can correspond to a third sensor of the robotic system 130 at a third position of the robotic system 130. For example, the right sensor 230 can be a visible light image sensor. The right sensor 230 can also be a time-of-flight sensor or depth sensor configured to capture at least one of intensity data or depth data in one or more visual spectra or non-visual spectra (e.g., infrared or ultraviolet) that depict one or more positions of one or more body parts of a surgeon at a console of the robotic device. Alternatively, the right sensor 230 may be a multi-modal sensor that includes one or more visible light image sensor and one or more depth sensors. For example, the right sensor 230 can capture image data or video data in one or more visual spectra or non-visual spectra (e.g., infrared or ultraviolet) that depict one or more positions of one or more body parts of a surgeon at the console of the robotic device. For example, the right sensor 230 can be coupled with the robotic system 130 at a right rail or arm of the robotic system facing the console of the robotic system 130. The right sensor 230 can detect the image data or the video data according to a third field of view. The third field of view can correspond to a physical volume within the medical environment 100B that is oriented toward the surgeon console and can be oriented to allow the right sensor 230 to detect the right hand manipulator 242 and optionally detect at least a portion of the left hand manipulator 240. For example, the third field of view can be oriented to allow the right sensor 230 to detect one or more hands, fingers, wrists, arms, forearms, or any portion thereof, engaged with the robotic system 130 or at least partially within the third field of view.

The left hand manipulator 240 can include one or more control affordances to modify position or state of one or more components of the robotic system 130 according to a medical procedure. For example, the left hand manipulator 240 can be structured to be grasped by a left hand and can include one or more moveable portions that can cause a robotic component of the robotic device to move. For example, the left hand manipulator 240 can be linked with a first robotic tool (e.g., cauterizing tool) to move, activate and deactivate the first robotic tool responsive to input received at the left hand manipulator 240 by a left hand or portion thereof (e.g., moving the left hand manipulator 240, pressing a button on the left hand manipulator 240, toggling a switch on the left hand manipulator 240) of the surgeon at the surgeon console. The right hand manipulator 242 can include one or more control affordances to modify position or state of one or more components of the robotic system 130 according to a medical procedure. For example, the right hand manipulator 242 can be structured to be grasped by a right hand and can include one or more moveable portions that can cause a robotic component of the robotic device to move. For example, the right hand manipulator 242 can be linked with a second robotic tool (e.g., forceps tool) to move, activate, and deactivate the second robotic tool responsive to input received at the right hand manipulator 242 by a right hand or portion thereof (e.g., moving the right hand manipulator 242, pressing a button on the right hand manipulator 242, toggling a switch on the right hand manipulator 242) of the surgeon at the surgeon console.

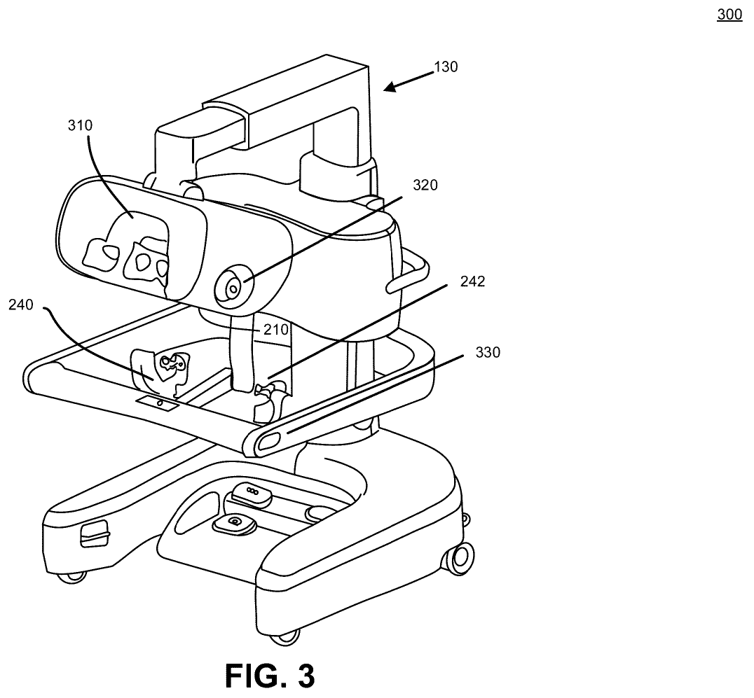

FIG. 3 depicts an example robotic system according to this disclosure. As illustrated by way of example in FIG. 3, a robotic system 300 can include at least a display system 310, an ergonomic head position controller 320, and an ergonomic arm position controller 330. For example, the robotic system 300 can correspond at least partially to the robotic system 130. For example, the robotic system 300 can include the robotic system 130 and one or more controllers that can be actuated to modify positions or orientations or one or more components of the robotic system 300 according to one or more linear directions (e.g., lift, lower, move forward, move backward, move left, or move right) or angular directions (e.g., pitch, yaw, or roll).

The display system 310 can correspond to a portion of the robotic system 300 that can generate or present at least a portion of the field of view 162, or a view of field captured via an instrument of the robotic system at the patient site. For example, the display system 310 can include two cameras that can collectively present a stereoscopic image of at least a portion of the patient site. For example, the center sensor 210 can be located behind the display system 310 on a center pillar of the robotic system 130. The ergonomic head position controller 320 can modify one or more of a position and orientation of the display system 310. For example, the ergonomic head position controller 320 can correspond to one or more control affordances (e.g., dials, buttons, switches) that can provide input to cause the robotic system 300 to move the display system 310. For example, the ergonomic head position controller 320 can cause the display system 310 to be moved according to one or more linear directions or angular directions. The ergonomic arm position controller 330 can modify one or more of a position and orientation of the robotic system 300 including the left hand manipulator 240 and the right hand manipulator 242. For example, the ergonomic arm position controller 330 can correspond to one or more control affordances (e.g., dials, buttons, or switches) that can provide input to cause the robotic system 300 to move a rail or arm holding the left hand manipulator 240 and the right hand manipulator 242. For example, the ergonomic arm position controller 330 can cause the rail or arm to be moved according to one or more linear directions or angular directions.

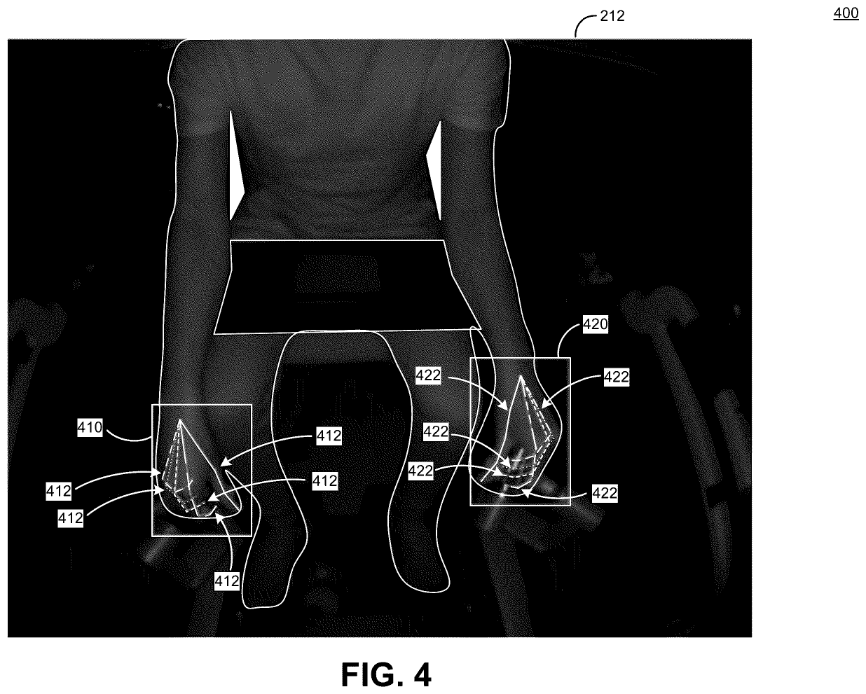

FIG. 4 depicts an example center camera field of view according to this disclosure. As illustrated by way of example in FIG. 4, a center camera field of view 400 can include at least a right hand capture region 410, and a left digit capture region 420. For example, the field of view 400 can correspond at least partially in one or more aspects of structure and operation to the field of view 212. For example, the center camera field of view 400 can correspond to an image or a frame of video including one or more images from the field of view 212. For example, the one or more images depict at least a portion of a left hand or a right hand engaged with one or more components of a robotic system or instrument, with the left hand or the right hand corresponding to the one or more body parts.

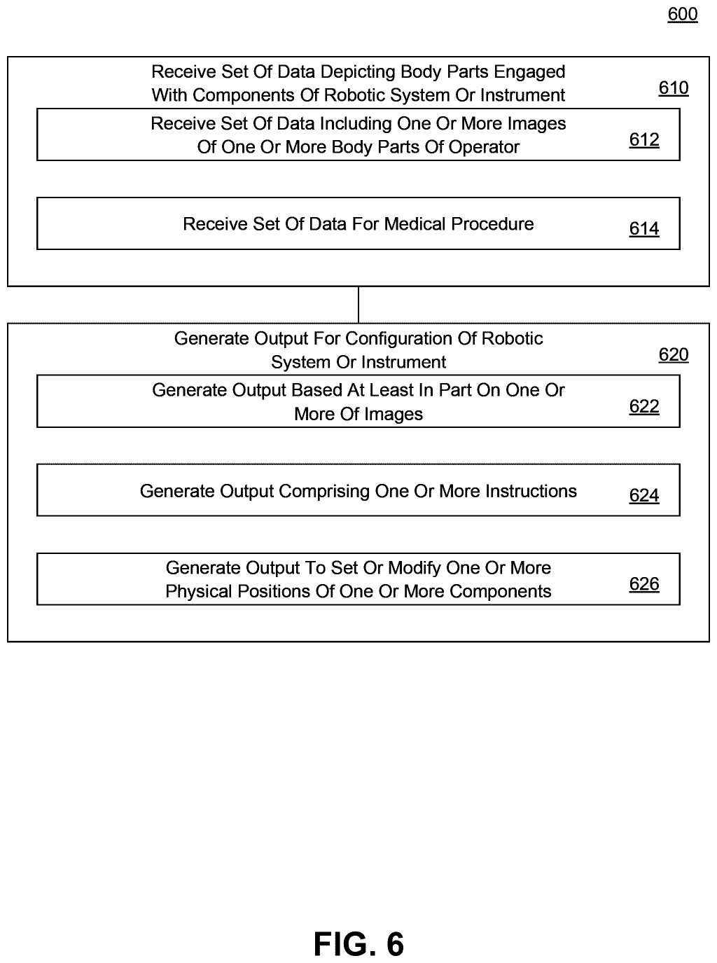

The right hand capture region 410 can correspond to a portion of the field of view 400 that at least partially contains an image corresponding to a right hand of a surgeon at the surgeon console. The right hand capture region 410 can include one or more wire frame models 412. For example, each wire frame model 412 can include one or more joint wire frames of the right hand, each indicative of respective portions of the respective digits of the right hand. For example, a first joint wire frame among the joint wire frames of the right hand can correspond to a right thumb of the right hand, a second joint wire frame among the joint wire frames of the right hand can correspond to a right index finger of the right hand, a third joint wire frame among the joint wire frames of the right hand can correspond to a right middle finger of the right hand, a fourth joint wire frame among the joint wire frames of the right hand can correspond to a right ring finger of the right hand, and a fifth joint wire frame among the joint wire frames of the right hand can correspond to a right little finger of the right hand. For example, the first joint wire frame among the joint wire frames of the right hand can identify one or more absolute positions or absolute orientations of a particular joint in a coordinate space. As discussed herein, a joint can correspond to a connection point between two distinct bones or a movement or rotation point between two stiff members (e.g., bones). For example, the model in FIG. 6 can generate one or more of the joint wire frames of the right hand based on input from one or more of the sensors 210, 220, and 230.

The left digit capture region 420 can correspond to a portion of the field of view 400 that at least partially contains an image corresponding to a left hand of a surgeon at the surgeon console. The left digit capture region 420 can include a left hand wire frame model 422. The left hand wire frame model 422 can include one or more joint wire frames of the left hand, each indicative of the respective portions of respective digits of the left hand. For example, a first joint wire frame among the joint wire frames of the left hand can correspond to a left thumb of the left hand, a second joint wire frame among the joint wire frames of the left hand can correspond to a left index finger of the left hand, a third joint wire frame among the joint wire frames of the left hand can correspond to a left middle finger of the left hand, a fourth joint wire frame among the joint wire frames of the left hand can correspond to a left ring finger of the left hand, and a fifth joint wire frame among the joint wire frames of the left hand can correspond to a left little finger of the left hand. For example, the first joint wire frame among the joint wire frames of the left hand can identify one or more absolute positions or absolute orientations of a particular joint in a coordinate space. As discussed herein, a joint can correspond to a connection point between two distinct bones or a movement or rotation point between two stiff members (e.g., bones). For example, the model in FIG. 6 can generate one or more of the joint wire frames of the left hand based on input from one or more of the sensors 210, 220, and 230.

The joint wire frames of the right and left hands can each correspond to one or more models, as discussed below, but are not limited thereto. For example, the one or more models each are indicative of the respective portions of the one or more body parts. For example, the models correspond to one or more wire frames each indicative of the respective physical positions of the respective portions of the one or more body parts. For example, the respective physical positions correspond to at least one of pitch, roll, or yaw of one or more of the respective portions of the one or more body parts.

The left hand or the right hand can correspond to portions of body parts as discussed herein. For example, the respective portions of the one or more body parts correspond to at least one joint of the one or more body parts. For example, the joint corresponds to at least one of a metacarpophalangeal joint, a proximal interphalangeal joint, and a distal interphalangeal joint. For example, the joint wire frame can be indicative of positions or orientations of one or more metacarpophalangeal joint, a proximal interphalangeal joint, and a distal interphalangeal joint.

For example, the system can generate, based at least in part on one or more of the images and one or more of the second images, a model indicative of a posture of the operator. The system can generate, based at least in part on the model, the output.

At least a system according to this disclosure can generate one or more metrics (e.g., quantitative values) corresponding to surgeon ergonomics, while interacting with the input system (i.e., surgeon console). In an aspect, the data processing system 110 can generate one or more metrics to identify one or more body positions of a surgeon at a surgeon console. For example, the system can provide one or more visual indications to the surgeon at a user interface display of the robotic system 104 during the surgical procedure in real time, to inform the surgeon of their ergonomic state and provide recommendations to modify the ergonomic state. For example, the system can provide one or more visual indications to the surgeon at a user interface display of a mobile or desktop computing device after the surgical procedure as an annotated video, to inform the surgeon of their ergonomic state and provide recommendations to modify the ergonomic state in or as part of an after-action report for the medical procedure.

For example, the data processing system 110 can generate one or more ergonomic metrics corresponding to objective performance indicators (an “OPI” or “OPIs”) of ergonomics of a surgeon over time (“ergonomic OPIs”). For example, ergonomic OPIs can be generated according to one or more aspects of a rapid upper limb assessment (“RULA”). For example, an ergonomic OPI can include a quantitative value based on one or more of an upper arm position, a lower arm position, a wrist position, a wrist twist, a muscle use, and a force or load affecting the arm or wrist. The system can generate the ergonomic API individually for each arm. For example, an ergonomic OPI can include a quantitative value based on one or more of a neck position, a trunk position, position, and a leg position. The system can generate the ergonomic API individually for each leg, and can modify an ergonomic OPI for the arm or wrist to generate an ergonomic OPI including one or more of the neck position, the trunk position, and the leg position.

In an aspect, the system can provide one or more ergonomic metrics or OPIs to a user interface, or cause a user interface to present one or more ergonomic metrics or OPIs. For example, the data processing system 110 can include a display device or be linked with a display device to present one or more ergonomic metrics or OPIs during a medical procedure (e.g., intra-operatively) at one or more times associated with the medical procedure. For example, the data processing system 110 can cause a user interface to notify a surgeon operating the robotic system 130 in real time, with one or more ergonomic metrics or OPIs indicative of non-optimal ergonomics of the surgeon at that moment. The data processing system 110 can, in response, generate one or more visual indications of recommendations to the surgeon to mitigate or eliminate the non-optimal ergonomics corresponding to the ergonomic metrics or OPIs. In an aspect, the system can cause a user interface to present an indication corresponding to the ergonomic metric.

In an aspect, the system can cause the user interface to present the indication during the action of the input system. For example, the data processing system 110 can generate and cause a user interface to present a recommendation to modify a pose, grip, or posture of the surgeon to mitigate or eliminate the non-optimal ergonomics corresponding to the ergonomic metrics or OPIs. For example, the data processing system 110 can generate and cause a user interface to present a recommendation to modify a state or configuration of the robotic system 130, or a portion thereof, to mitigate or eliminate the non-optimal ergonomics corresponding to the ergonomic metrics or OPIs. For example, the data processing system 110 can generate and cause a user interface to present a recommendation to clutch one or more of the manipulators. For example, the data processing system 110 can generate and cause a user interface to present a recommendation to re-center a workspace of the robotic system 130 with respect to at least one of the body of the operator (e.g., surgeon). Thus, the data processing system 110 can generate user interface output including visual indications to improve surgeon ergonomics at a level of granularity and real-time responsiveness beyond the capability of manual processes to achieve.

For example, the data processing system 110 can include a display device or be linked with a display device to present one or more ergonomic metrics or OPIs after a medical procedure (e.g., post-operatively) at one or more times associated with the medical procedure. For example, the data processing system 110 can generate a video having one or more annotations, or one or more annotations associated with one or timestamps and ergonomic metrics. In an aspect, the indication includes a visual output at the user interface. In an aspect, the system can determine that the ergonomic metric satisfies a threshold at one or more times, the threshold indicative of a type of pose for the body of the operator. In an aspect, the system can cause the user interface to present the indication at the one or more times during the action of the input system. In an aspect, the system can cause the user interface to present the indication indicative of the one or more times. In an aspect, the system can identify, based at least partially on the plurality of values, a second plurality of values representing second joint angles of the one or more joints of the body of the operator. In an aspect, the system can generate, based at least partially on the second plurality of values, one or more second kinematic representations of the body of the operator.

In an aspect, the system can cause a user interface to present an indication including a recommendation to modify a first pose of the body of the operator to a second pose of the body of the operator, the first pose corresponding to the one or more kinematic representations, and the second pose corresponding to the one or more second kinematic representations. For example, the system can generate post-operative recommendations corresponding to a configuration of a robotic system for a given surgeon with respect to a given robotic system and a given medical procedure. For example, the system can generate a recommendation to eliminate non-optimal ergonomics with respect to a given surgeon with respect a given medical procedure, based on kinematic representations of the given surgeon performing one or more instances of the medical procedure. For example, the system can generate a recommendation to eliminate non-optimal ergonomics with respect to a given surgeon with respect a given medical procedure, based on operator information, including but not limited to metrics of the given surgeon during for prior medical procedures, preference of the given surgeon with respect to the robotic system 130, physiology of the surgeon, including biomechanical parameters as discussed herein, or any combination thereof. For example, the system can generate a recommendation to modify a configuration of the robotic system or location or presence of one or more objects in a medical environment associated with a given medical procedure, with respect to a given surgeon with respect a given medical procedure, based one or more of the kinematic representations, the biomechanical parameters, or the occlusion parameters, as discussed herein. Though discussed herein with respect to a medical procedure, this technical solution is not limited to generating recommendations with respect to an entire medical procedure. For example, the system can generate recommendations specific to a given phase of a medical procedure, or a task of a phase of a medical procedure, based on training input that identifies a task or phase, or training input that is limited to a given task or phase, but is not limited thereto.

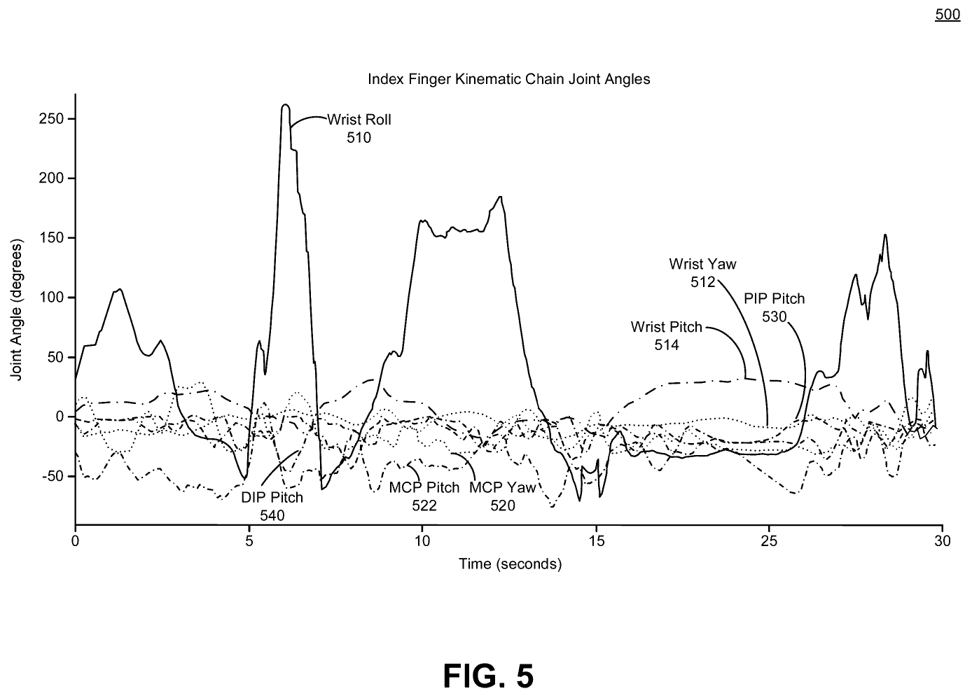

FIG. 5 depicts an example set of joint angle data generated for a digit of an operator of a medical system according to this disclosure. The set of joint angle data may correspond to joint angles of each joint of a kinematic chain representing the digit of the operator and a plurality of sets of joint angle data (e.g., a set for each respective digit) may be generated for the operator as the operator operates the medical system. According to embodiments, multiple time-series data relating to a particular joint of the digit may be included in the set of joint angle data. Each time-series data corresponding the joint may represent joint angles over time of a respective degree of freedom (e.g., pitch, jaw, roll) of the particular joint. Furthermore, as depicted in FIG. 5, the set of joint angle data may include angle data relating to one or more shared joints (e.g., wrist joint) common to kinematic chains of other digits of the operator.

According to embodiments, the set of joint angle data may be generated using one or more pose estimation models and/or joint kinematic models based on sensor data (e.g., visible image data, depth data, etc.) captured of the operator. Referring to FIG. 4, the set of joint angle data depicted in FIG. 5 may be generated using the wire frame models 412 or 422. In particular, the pose estimation model may receive an image frame captured by the sensors configured to detect the operator's pose (e.g., visible light image sensors of sensors 210, 220, 230) to identify key features (e.g., joints for which angle data are to be captured) on the operator's hand and provide an estimate two-dimensional positions (e.g., in 2D image coordinate space) of the key features in the image frame. The pose estimation model (or another spatial positioning model) may further receive a depth frame (e.g., depth data or point cloud data for a particular time frame) captured by sensors configured to capture depth information relating the operator's pose (e.g., time-of-flight sensors, depth sensors, etc. of sensors 210, 220, 230) to determine positions of the key features of the operator's hand in three-dimensional space. The generated three-dimensional positions of the key features (e.g., joints of the operator's hand) may be received by one or more joint kinematic models representing the operator's hand. The joint kinematic models may define biomechanically feasible configurations of the operator's hand and may generate, using the three-dimensional positions of the key features, sets of joint angle data such as the one depicted in FIG. 5.

According to embodiments, the sets of joint angle data, which include time series-data of various degrees of freedoms of joints of the hand of the operator, may be used by the system to generate metrics (e.g., ergonomic OPIs) relating to the operator, as described throughout this disclosure. Furthermore, although FIG. 5 describes joint angle data for a digit and/or a hand of the operator, it is understood that the techniques for generating data relating to the pose of the operator is not limited to being applied to joints of the hand of the operator. For instance, it is understood that data relating to, for example, an elbow pose, a shoulder pose, a neck pose, and the like may be generated in a similar manner that is described herein.

In more detail, the set of joint angle data generated for a digit 500 can include at least a wrist roll feature 510, a wrist yaw feature 512, a wrist pitch feature 514, a metacarpophalangeal (MCP) yaw feature 520, an MCP pitch feature 522, a proximal interphalangeal (PIP) pitch feature 530, and a distal interphalangeal (DIP) pitch feature 540. Each of the features described herein with respect to FIG. 5 may be time-series data representing joint angles generated for a particular joint relating to a particular degree of freedom of the particular joint. For example, the kinematic capture model for a digit 500 can correspond to the second joint wire frame among the joint wire frames of the right hand that corresponds to the right index finger of the right hand. For example, the kinematic capture model for a digit 500 can correspond to the second joint wire frame among the joint wire frames of the left hand that corresponds to the left index finger of the right hand.

The wrist roll feature 510 can be indicative of a first angular measurement of a first body part associated with the second joint wire frame. For example, the first angular measurement can correspond to roll. For example, the first body part can correspond to a left wrist for second joint wire frame for the left hand, or can correspond to a right wrist for second joint wire frame for the right hand. This technical solution thus includes a technical improvement to determine, according to a machine learning model as discussed herein, a first angular measurement of a first body part external to a digit, according to a joint wire frame corresponding to the digit. The wrist yaw feature 512 can be indicative of a second angular measurement of the first body part associated with the second joint wire frame. For example, the second angular measurement can correspond to yaw. This technical solution thus includes a technical improvement to determine, according to a machine learning model as discussed herein, a second angular measurement of a first body part external to a digit, according to a joint wire frame corresponding to the digit. The wrist pitch feature 514 can be indicative of a third angular measurement of the first body part associated with the second joint wire frame. For example, the third angular measurement can correspond to pitch. This technical solution thus includes a technical improvement to determine, according to a machine learning model as discussed herein, a third angular measurement of a first body part external to a digit, according to a joint wire frame corresponding to the digit.

The metacarpophalangeal (MCP) yaw feature 520 can be indicative of a first angular measurement of a first portion of the first body part associated with the second joint wire frame. For example, the first angular measurement of the first portion can correspond to yaw. For example, the first portion of the first body part can correspond to an MCP joint. This technical solution thus includes a technical improvement to determine, according to a machine learning model as discussed herein, a first angular measurement of a first portion of a first body part corresponding to a digit, according to a joint wire frame corresponding to the digit.

The MCP pitch feature 522 can be indicative of a second angular measurement of the first portion of the first body part associated with the second joint wire frame. For example, the second angular measurement of the first portion can correspond to pitch. This technical solution thus includes a technical improvement to determine, according to a machine learning model as discussed herein, a second angular measurement of a first portion of a first body part corresponding to a digit, according to a joint wire frame corresponding to the digit.

The proximal interphalangeal (PIP) pitch feature 530 can be indicative of an angular measurement of a second portion of the first body part associated with the second joint wire frame. For example, the angular measurement of the second portion can correspond to pitch. For example, the second portion of the first body part can correspond to a PIP joint. This technical solution thus includes a technical improvement to determine, according to a machine learning model as discussed herein, an angular measurement of a second portion of a first body part corresponding to a digit, according to a joint wire frame corresponding to the digit.

The distal interphalangeal (DIP) pitch feature 540 can be indicative of an angular measurement of a third portion of the first body part associated with the second joint wire frame. For example, the angular measurement of the third portion can correspond to pitch. For example, the third portion of the first body part can correspond to a DIP joint. This technical solution thus includes a technical improvement to determine, according to a machine learning model as discussed herein, an angular measurement of a third portion of a first body part corresponding to a digit, according to a joint wire frame corresponding to the digit. Thus, the model can determine multiple angular measurements for multiple joints of a digit based on a wireframe corresponding to the digit.

In an aspect, the system can measure changes in various joint angles over time using time series data to determine the ergonomic metrics. For example, each of the angular measurements discussed above can correspond to particular times (e.g., timestamps) associated with the medical procedure, indicative of times at which the sensors (e.g., time-of-flight sensors) captured a state of a hand or one or more digits for the angular measurements. For example, the system can determine an ergonomic metric or OPI with respect to time or over a period of time, including or more of a displacement, a rate of change of displacement (e.g., a first derivative of displacement), an acceleration with respect to displacement (e.g., a second derivative of displacement), or a jerk with respect to displacement (e.g., a third derivative of displacement). For example, the system can determine an ergonomic metric corresponding to one or more of the displacement or any derivative thereof, to provide a technical improvement to identify ergonomic states at a granularity beyond the capability of manual processes to achieve. Thus, the ergonomic OPIs can be indicative of ergonomic data beyond position or location.

In an aspect, the system can fuse output from multiple sensors, including, for example, fusing output from multiple time-of-flight sensors in the medical environment. For example, outputs respectively corresponding to individual time-of-flight sensors can be fused according to one or more features of the outputs indicative of the sensor data from each of the time-of-flight sensors and structured as input to a machine learning model configured to receive image features as input. For example, the system can fuse one or more of the outputs, and can provide one or more of the fused outputs to the machine learning model to generate the joint wire frame or a portion thereof. For example, the system can weight one or more of the outputs, and can fuse one or more of the weighted outputs, and can provide one or more of the fused and weighted outputs to the machine learning model to generate the joint wire frame or a portion thereof. For example, the joint wire frame or a portion thereof can correspond to a kinematic representation of at least a portion of a body of an operator, as discussed herein.

The system can generate weights according to one or more parameters. For example, the system can detect one or more biomechanical parameters associated with the body of the surgeon, and can generate one or more weights sensor data from one or more sensors based on one or more of the biomechanical parameters. For example, a machine learning model configured to detect image features can identify one or more biomechanical parameters including hand size, permissible joint angles, or other biomechanical constraints of a hand of a given surgeon according to one or more biomechanical properties of a body part of the surgeon at least as discussed herein. For example, a first time-of-flight sensor can have a field of view from a first position that can detect a joint angle with clarity (e.g., a side view of a digit). For example, a second time-of-flight sensor can have a field of view from a first position that can detect a joint angle with low clarity (e.g., a back view of a digit, where angle detection is not apparent). The system can apply a greater weight to the sensor data of the first time-of-flight sensor, in accordance with a determination that the first time-of-flight sensor can detect the biomechanical parameter for permissible joint angles with high confidence. The system can apply a lesser weight to the sensor data of the second time-of-flight sensor, in accordance with a determination that the second time-of-flight sensor cannot detect the biomechanical parameter with high confidence.

For example, the system can determine one or more weights according to a determination of occlusion of one or more portions of the body of the surgeon across one or more sensors. For example, a machine learning model configured to detect image features can identify one or more occlusion parameters including lack of visibility of one or more digits, portions of digits, hands, portions of hands, or any combination thereof, from one or more fields of views associated with corresponding sensors. For example, a first time-of-flight sensor can have a field of view from a first position that can view all joints of a first digit (e.g., an unobstructed view of the first digit). For example, a second time-of-flight sensor can have a field of view from a second position that can view only a subset of joints of the first digit (e.g., an obstructed view of the first digit). The system can apply a greater weight to the sensor data of the first time-of-flight sensor, in accordance with a determination that the first time-of-flight sensor is associated with an occlusion parameter that indicates that the first time-of-flight sensor can detect all joints of the first digit with high confidence. The system can apply a lesser weight to the sensor data of the second time-of-flight sensor, in accordance with a determination that the second time-of-flight sensor is associated with an occlusion parameter that indicates that the second time-of-flight sensor cannot detect all joints of the first digit with high confidence. The occlusion parameters as discussed herein are not limited to any one digit or portion of a body of the surgeon as discussed herein by way of example, and can apply at least to any number of digits or portions of a hand, arm, torso, neck, leg, or any combination thereof. Thus, the system can perform actions including fusing and weighing data from the multiple sensors to obtain hand/body kinematic information, based on biomechanical parameters, occlusion parameters, or both. The fusing and weighting as discussed herein can provide a technical improvement that is superior to using probabilistic processes for hand tracking.

For example, the one or more physical positions of the one or more body parts each correspond to respective poses of the one or more body parts engaged with the one or more components of the robotic system or instrument. For example, respective poses can include a slouched position of a surgeon, an upright sitting position of a surgeon, a grip with a straight wrist in line with a manipulator, a grip turned inward with respect to a manipulator, or any combination thereof. Thus, the cameras as discussed herein can determine one or more of positions of one or more body parts of a surgeon including, but not limited to digits, wrists, arms, forearms, shoulders, upper back, lower back, or any portion thereof, or any combination thereof.

For example, the first model is indicative of the respective portions of the one or more body parts. For example, the first model corresponds to one or more wire frames each indicative of the respective physical positions of the respective portions of the one or more body parts. For example, a machine learning model at least as discussed herein can generate the wire frames by aggregating input from one or more images or video. For example, the first model is indicative of respective portions of the one or more body parts of the operator, and where the respective portions of the one or more body parts correspond to at least one joint of the one or more body parts. For example, the joint corresponds to at least one of a metacarpophalangeal joint, a proximal interphalangeal joint, and a distal interphalangeal joint.

For example, the one or more images can depict at least a portion of a left hand or a right hand engaged with one or more components of a robotic system or instrument, the left hand or the right hand corresponding to the one or more body parts. For example, the system can receive a second set of data that can include one or more of the second images of the one or more body parts of the operator, the one or more second images corresponding to a viewpoint of the operator distinct from a second viewpoint of the operator corresponding to the set of data. sensor 210sensor 220

FIG. 6 depicts an example method of configuration of robotic systems for ergonomic states of operators in medical procedures according to this disclosure. At least one of the system 100A, the robotic system 130, or the robotic system 300can perform method 600. At 610, the method 600 can receive a set of data depicting body parts engaged with components of a robotic system or instrument. For example, the method can include receiving a second set of data that can include one or more second images of the one or more body parts of the operator, the one or more second images corresponding to a viewpoint of the operator distinct from a second viewpoint of the operator corresponding to set of data. At 612, the method 600 can receive the set of data including one or more images of one or more body parts of an operator. At 614, the method 600 can receive the set of data for a medical procedure.

At 620, the method 600 can generate an output for a configuration of the robotic system or instrument. For example, the method can include generating, based at least in part on the one or more images, one or more models each indicative of respective portions of the one or more body parts of the operator. The method can include generating, based at least in part on the model, the output. For example, the one or more models each can be indicative of the respective portions of the one or more body parts. For example, the models can correspond to one or more wire frames each indicative of the respective physical positions of the respective portions of the one or more body parts, and where the respective physical positions correspond to at least one of pitch, roll, or yaw of one or more of the respective portions of the one or more body parts. For example, the method can generate, based at least in part on one or more of the images and one or more of the second images, a model indicative of a posture of the operator. The method can include generate, based at least in part on the model, the output. At 622, the method 600 can generate the output based at least in part on one or more of the images. For example, the one or more images can depict at least a portion of a left hand or a right hand engaged with one or more components of a robotic system or instrument, the left hand or the right hand corresponding to the one or more body parts. For example, the respective portions of the one or more body parts correspond to at least one joint of the one or more body parts, and where the joint corresponds to at least one of a metacarpophalangeal joint, a proximal interphalangeal joint, and a distal interphalangeal joint. At 624, the method 600 can generate the output comprising one or more instructions. At 626, the method 600 can generate the output to set or modify one or more physical positions of the one or more components.

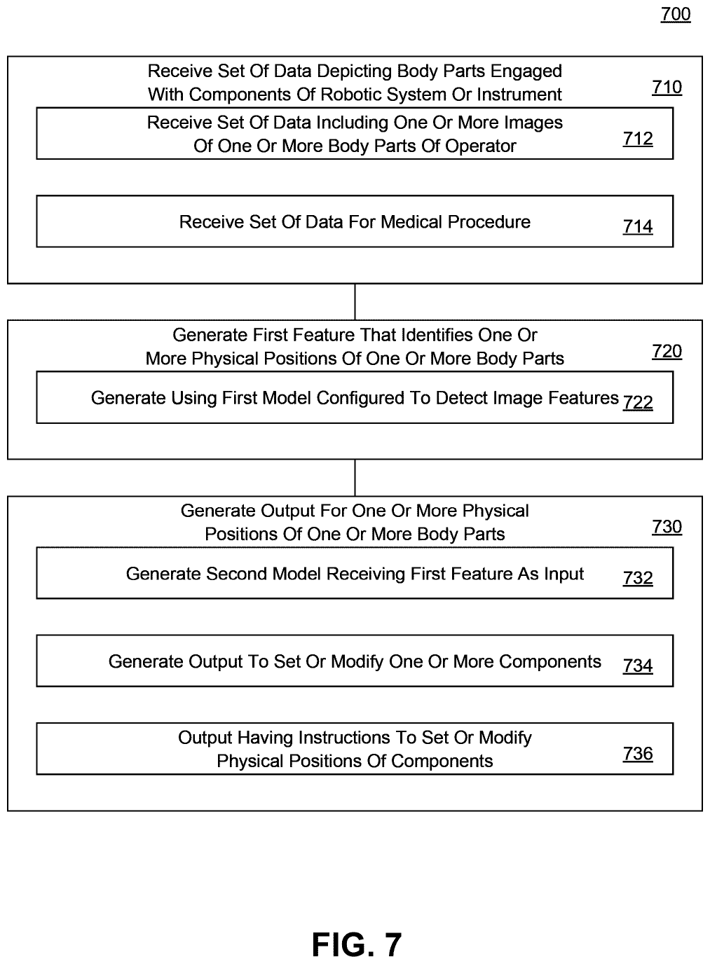

FIG. 7 depicts an example method of configuration of robotic systems for ergonomic states of operators in medical procedures according to this disclosure. At least one of the system 100A, the robotic system 130, or the robotic system 300can perform method 700. At 710, the method 700 can receive a set of data depicting body parts engaged with components of a robotic system or instrument. At 712, the method 700 can receive the set of data including one or more images of one or more body parts of an operator. For example, the one or more images depict at least a portion of a left hand or a right hand engaged with one or more components of a robotic system or instrument, the left hand or the right hand corresponding to the one or more body parts. For example, the one or more physical positions of the one or more body parts each correspond to respective poses of the one or more body parts engaged with the one or more components of the robotic system or instrument. At 714, the method 700 can receive the set of data for a medical procedure.

At 720, the method 700 can generate a first feature that identifies one or more physical positions of the one or more body parts. At 722, the method 700 can generate the first feature using a first model configured to detect image features. At 730, the method 700 can generate an output for the one or more physical positions of the one or more body parts. At 732, the method 700 can generate a second model receiving the first feature as input. At 734, the method 700 can generate the output to set or modify the one or more components. For example, the method can include generating, based at least in part on one or more of the images and one or more of the second images, a model indicative of a posture of the operator. The method can include generating, based at least in part on the model, the output. At 736, the method 700 can generate the output having instructions to set or modify physical positions of the components.

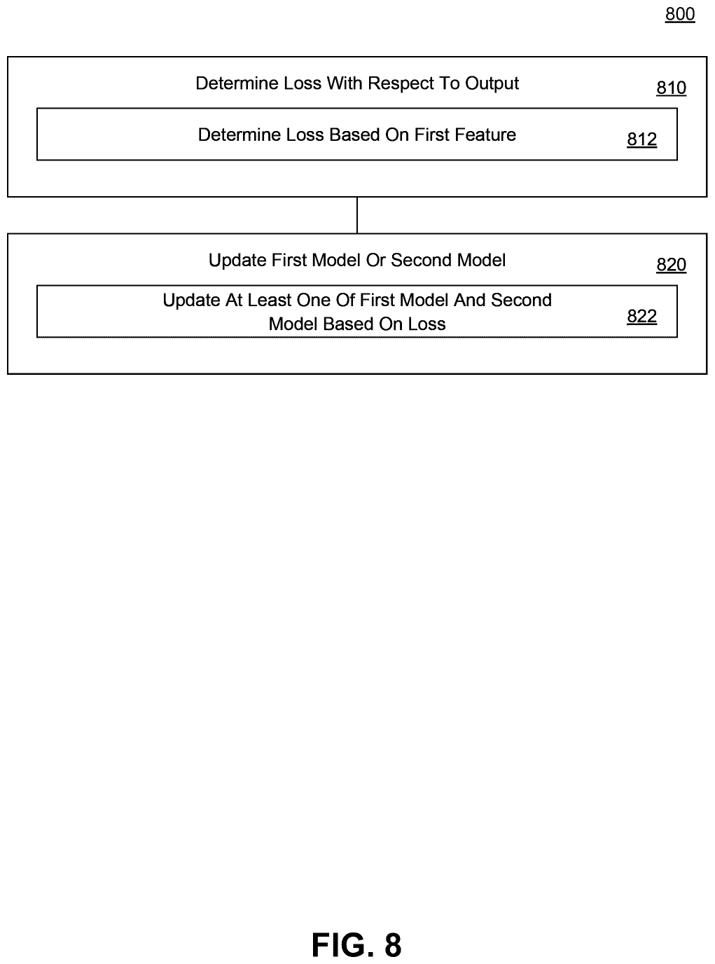

FIG. 8 depicts an example method of configuration of robotic systems for ergonomic states of operators in medical procedures according to this disclosure. At least one of the system 100A, the robotic system 130, or the robotic system 300can perform method 800. At 810, the method 800 can determine a loss with respect to the output. At 812, the method 800 can determine the loss based on the first feature. For example, the system 100A can determine the loss based on one or more of the features of FIG. 5 and the image data corresponding to the features of FIG. 5. For example, the method can include receiving a second set of data, can include one or more of the second images of the one or more body parts of the operator, the one or more second images corresponding to a viewpoint of the operator distinct from a second viewpoint of the operator corresponding to set of data.

At 820, the method 800 can update the first model or the second model. For example, the first model corresponds to one or more wire frames each indicative of the respective physical positions of the respective portions of the one or more body parts, and where the respective physical positions correspond to at least one of pitch, roll, or yaw of one or more of the respective portions of the one or more body parts. For example, the first model is indicative of respective portions of the one or more body parts of the operator, and where the respective portions of the one or more body parts correspond to at least one joint of the one or more body parts, and where the joint corresponds to at least one of a metacarpophalangeal joint, a proximal interphalangeal joint, and a distal interphalangeal joint. For example, the method 800 can update the first model or the second model based on a feedback input including at least one of the wire frames as discussed herein. At 822, the method 800 can update at least one of the first model and the second model based on the loss.

Having now described some illustrative implementations, the foregoing is illustrative and not limiting, having been presented by way of example. In particular, although many of the examples presented herein involve specific combinations of method acts or system elements, those acts and those elements may be combined in other ways to accomplish the same objectives. Acts, elements and features discussed in connection with one implementation are not intended to be excluded from a similar role in other implementations.

The phraseology and terminology used herein is for the purpose of description and should not be regarded as limiting. The use of “including,” “comprising,” “having,” “containing,” “involving,” “characterized by,” “characterized in that,” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

References to “or” may be construed as inclusive so that any terms described using “or” may indicate any of a single, more than one, and all of the described terms. References to at least one of a conjunctive list of terms may be construed as an inclusive OR to indicate any of a single, more than one, and all of the described terms. For example, a reference to “at least one of ‘A’ and ‘B’” can include only ‘A’, only ‘B’, as well as both ‘A’ and ‘B’. Such references used in conjunction with “comprising” or other open terminology can include additional items. References to “is” or “are” may be construed as nonlimiting to the implementation or action referenced in connection with that term. The terms “is” or “are” or any tense or derivative thereof, are interchangeable and synonymous with “can be” as used herein, unless stated otherwise herein.

Directional indicators depicted herein are example directions to facilitate understanding of the examples discussed herein, and are not limited to the directional indicators depicted herein. Any directional indicator depicted herein can be modified to the reverse direction, or can be modified to include both the depicted direction and a direction reverse to the depicted direction, unless stated otherwise herein. While operations are depicted in the drawings in a particular order, such operations are not required to be performed in the particular order shown or in sequential order, and all illustrated operations are not required to be performed. Actions described herein can be performed in a different order. Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Scope of the systems and methods described herein is thus indicated by the appended claims, rather than the foregoing description. The scope of the claims include equivalents to the meaning and scope of the appended claims.

What is claimed is:

1. A system, comprising:
one or more processors, coupled with memory, to:
receive a set of data including one or more images of one or more body parts of an operator, the one or more images depicting the one or more body parts engaged with one or more components of a robotic system or instrument, the set of data corresponding to a medical procedure; and
generate, based at least in part on one or more of the images, an output corresponding to a configuration of the robotic system or instrument, the output comprising one or more instructions to set or modify one or more physical positions of the one or more components.

2. The system of claim 1, the processors to:
generate, based at least in part on the one or more images, one or more models each indicative of respective portions of the one or more body parts of the operator; and
generate, based at least in part on the model, the output, wherein the one or more models each are indicative of the respective portions of the one or more body parts, wherein the models correspond to one or more wire frames each indicative of the respective physical positions of the respective portions of the one or more body parts, wherein the respective physical positions correspond to at least one of pitch, roll, or yaw of one or more of the respective portions of the one or more body parts, wherein the respective portions of the one or more body parts correspond to at least one joint of the one or more body parts, and wherein the joint corresponds to at least one of a metacarpophalangeal joint, a proximal interphalangeal joint, and a distal interphalangeal joint.

3. The system of claim 1, wherein the one or more images depict at least a portion of a left hand or a right hand engaged with one or more components of a robotic system or instrument, the left hand or the right hand corresponding to the one or more body parts.

4. The system of claim 1, the processors to:
receiving a second set of data including one or more second images of the one or more body parts of the operator, the one or more second images corresponding to a viewpoint of the operator distinct from a second viewpoint of the operator corresponding to set of data;
generate, based at least in part on one or more of the images and one or more of the second images, a model indicative of a posture of the operator; and
generate, based at least in part on the model, the output.

5. A system, comprising:
one or more processors, coupled with memory, to:
receive data comprising one or more images of one or more body parts of an operator, the one or more images depicting the one or more body parts engaged with one or more components of a robotic system or instrument, the set of data corresponding to a medical procedure;
generate, using a first model configured to detect image features, a first feature that identifies one or more physical positions of the one or more body parts;
generate, using a second model receiving the first feature as input, an output corresponding to the one or more physical positions of the one or more body parts, the output comprising one or more instructions to set or modify one or more physical positions of the one or more components;
determine, based on the first feature, a loss with respect to the output; and
update at least one of the first model and the second model based on the loss.

6. The system of claim 5, wherein the one or more physical positions of the one or more body parts each correspond to respective poses of the one or more body parts engaged with the one or more components of the robotic system or instrument.

7. The system of claim 5, wherein the first model is indicative of the respective portions of the one or more body parts.

8. The system of claim 7, wherein
the first model corresponds to one or more wire frames each indicative of the respective physical positions of the respective portions of the one or more body parts; and
the respective physical positions correspond to at least one of pitch, roll, or yaw of one or more of the respective portions of the one or more body parts.

9. The system of claim 5, wherein
the first model is indicative of respective portions of the one or more body parts of the operator, and wherein the respective portions of the one or more body parts correspond to at least one joint of the one or more body parts; and
the joint corresponds to at least one of a metacarpophalangeal joint, a proximal interphalangeal joint, and a distal interphalangeal joint.

10. The system of claim 5, wherein the one or more images depict at least a portion of a left hand or a right hand engaged with one or more components of a robotic system or instrument, the left hand or the right hand corresponding to the one or more body parts.

11. The system of claim 5, the processors to:
receiving a second set of data including one or more second images of the one or more body parts of the operator, the one or more second images corresponding to a viewpoint of the operator distinct from a second viewpoint of the operator corresponding to set of data;
generate, based at least in part on one or more of the images and one or more of the second images, a model indicative of a posture of the operator; and
generate, based at least in part on the model, the output.

12. A medical system, comprising:
a manipulator assembly configured to support one or more medical instruments; an input system configured to be operated by an operator to control the manipulator assembly, wherein the input system includes an operator workspace;
a sensing system comprising one or more sensors, the one or more sensors having a combined field of view of at least a portion of the operator workspace;
one or more processors, coupled with memory to:
receive sensor data from the one or more sensors of the sensing system;
generate, based on the sensor data, one or more kinematic representations of at least a portion of a body of the operator;
compute, based on the one or more kinematic representations, a plurality of values, the plurality of values representing joint angles of one or more joints of the body of the operator; and
determine, based on the plurality of values, an ergonomic metric for an action of the input system via the operator.

13. The medical system of claim 12, wherein the sensing system includes at least one depth sensor, and wherein the sensor data includes depth data from the at least one of depth sensor that is at least partially indicative of the one or more kinematic representations.

14. The medical system of claim 12, wherein the sensing system includes at least one time-of-flight sensor, and wherein the sensor data includes time-of-flight data from the at least one of time-of-flight sensor that is at least partially indicative of the one or more kinematic representations.

15. The medical system of claim 12, the one or more processors to:
fuse one or more features of the sensor data into one or more fused features; and
generate, according to the one or more fused features, the one or more kinematic representations.

16. The medical system of claim 12, the one or more processors to:
generate, one or more weights according to one or more fields of view respectively corresponding to each of the one or more sensors; and
fuse, based on the one or more weights, the sensor data into one or more fused features.

17. The medical system of claim 12, the one or more processors to:
cause a user interface to present an indication corresponding to the ergonomic metric;
cause the user interface to present the indication during the action of the input system; and
the indication includes a visual output at the user interface;

18. The medical system of claim 12, the one or more processors to:
determine that the ergonomic metric satisfies a threshold at one or more times, the threshold indicative of a type of pose for the body of the operator;
cause the user interface to present the indication at the one or more times during the action of the input system; and
cause the user interface to present the indication indicative of the one or more times.

19. The medical system of claim 12, the one or more processors to:
identify, based at least partially on the plurality of values, a second plurality of values representing second joint angles of the one or more joints of the body of the operator; and
generate, based at least partially on the second plurality of values, one or more second kinematic representations of the body of the operator.

20. The medical system of claim 19, the one or more processors to:
cause a user interface to present an indication including a recommendation to modify a first pose of the body of the operator to a second pose of the body of the operator, the first pose corresponding to the one or more kinematic representations, and the second pose corresponding to the one or more second kinematic representations.
